(19) United States
(12) Patent Application Publication
(10) Pub. No.: US 2021/0340602 A1
(43) Pub. Date: Nov. 4, 2021

(54) SINGLE-MOLECULE EPIGENETIC LOCALIZATION

(71) Applicants: UNIVERSITY OF CINCINNATI, Cincinnati, OH (US); CHANCELLOR, MASTERS AND SCHOLARS OF THE UNVIVERSITY OF OXFORD, Oxford (GB)

(72) Inventors: Jiajie Diao, Cincinnati, OH (US); Chunxiao Song, Oxford (GB)

(21) Appl. No.: 17/283,378

(22) PCT Filed: Oct. 16, 2019
(86) PCT No.: PCT/US2019/056439

§ 371 Date: Apr. 7, 2021

Related U.S. Application Data
(60) Provisional application No. 62/746,121, filed on Oct. 16, 2018.

(51) Int. Cl. C12Q 1/6827; C12Q 1/6886; G01N 21/64

(52) U.S. Cl. CPC C12Q 1/6827; C12Q 1/6886; G01N 21/6428; G01N 2201/06113; G01N 21/6402; C12Q 2600/154; G01N 2021/6441; G01N 21/6458

(57) ABSTRACT

A method for localizing epigenetic modifications of DNA is provided, including: providing a target DNA strand having a least one epigenetic modification, wherein the target DNA strand is annealed to a non-target DNA stand, wherein each of the target DNA strand and the non-target DNA strand is labeled with a first fluorophore; labeling the at least one epigenetic modification with a second fluorophore; annealing a first probe to the target DNA strand and annealing a second probe to the non-target DNA strand; immobilizing the target DNA strand on a support; and detecting the first and second fluorophores immobilized on the support. Also provided is a method of diagnosing a disease or condition, such as cancer, in a subject suspected of having the disease by localizing epigenetic modifications of DNA from a patient sample and comparing to a reference epigenetic profile associated with the disease or condition.

### CROSS-REFERENCE TO RELATED APPLICATIONS

This application claims priority to U.S. Provisional Application Ser. No. 62/746,121, filed Oct. 16, 2018, which is incorporated by reference herein in its entirety.

### SEQUENCE LISTING

Applicant incorporates by reference a CRF sequence listing submitted herewith having file name Sequence_Listing_10738_758.txt, created on Oct. 4, 2019.

The nucleic acid sequences listed in the accompanying sequence listing are shown using standard abbreviations as defined in 37 C.F.R. 1.822. In the accompanying sequence listing:

SEQ ID NO: 1 represents a target DNA strand;

SEQ ID NO: 2 represents a non-target DNA strand;

SEQ ID NO: 3 represents a single-stranded DNA probe complementary to a target DNA strand;

SEQ ID NO: 4 represents a single-stranded DNA probe complementary to a non-target DNA strand;

SEQ ID NO: 5 represents a target DNA strand lacking 5hmC modification;

SEQ ID NO: 6 represents a single-stranded DNA probe complementary to a target DNA strand SP;

SEQ ID NO: 7 represents a non-target DNA strand;

SEQ ID NO: 8 represents a single-stranded DNA probe complementary to a non-target DNA strand SP2;

SEQ ID NO: 9 represents a target DNA strand having one 5hmC modification;

SEQ ID NO: 10 represents a single-stranded DNA probe complementary to a target DNA strand SP3;

SEQ ID NO: 11 represents a non-target DNA strand;

SEQ ID NO: 12 represents a single-stranded DNA probe complementary to a non-target DNA strand SP4;

SEQ ID NO: 13 represents a target DNA strand having two 5hmC modifications;

SEQ ID NO: 14 represents a single-stranded DNA probe complementary to a target DNA strand SP5;

SEQ ID NO: 15 represents a non-target DNA strand;

SEQ ID NO: 16 represents a single-stranded DNA probe complementary to a non-target DNA strand SP6;

SEQ ID NO: 17 represents a target DNA strand having three 5hmC modifications;

SEQ ID NO: 18 represents a single-stranded DNA probe complementary to a target DNA strand SP7;

SEQ ID NO: 19 represents a non-target DNA strand;

SEQ ID NO: 20 represents a single-stranded DNA probe complementary to a non-target DNA strand SP8;

SEQ ID NO: 21 represents a target DNA strand;

SEQ ID NO: 22 represents a single-stranded DNA probe complementary to a target DNA strand SP-a;

SEQ ID NO: 23 represents a non-target DNA strand; and

SEQ ID NO: 24 represents a single-stranded DNA probe complementary to a non-target DNA strand SP-b.

### BACKGROUND

DNA epigenetic modifications play important functions in a broad range of physiological and pathological processes and their dysregulation can lead to various human diseases. 5-hydroxymethylcytosine (5hmC), one of the major mammalian DNA epigenetic modifications, is generated by ten-eleven translocation (TET) family proteins from 5-methylcytosine (5mC) and is often referred to as the sixth base of DNA, due to its involvement in epigenetic reprogramming and regulation of gene expression. 5hmC is tissue-specific and is believed to be a gene activation marker in development and disease. Recently, 5hmC has been reported as an epigenetic biomarker for several types of cancer.

Circulating cell-free DNA (cfDNA) are short, degraded nucleic acid fragments in circulation in the bloodstream. The non-invasive availability of cfDNA makes it a promising biomarker for diagnosing, prognosing, and monitoring tumor evolution and response to therapy. Using a sensitive chemical labeling-based low-input sequencing method, the present investigators previously conducted rapid and reliable sequencing of 5hmC in cfDNA and showed that cell-free 5hmC displays distinct features in several types of cancer. Song, et al., 5-Hydroxymethylcytosine signatures in cell-free DNA provide information about tumor types and stages, Cell Res. 27(10): 1231-42 (2017). These findings have potential application not only in identifying cancer types, but also in diagnosis of cancer and tracking tumor stage in some cancers. In order to work with the minute quantities of cfDNA available (typically only a few nanograms per ml of plasma), ultra-sensitive detection methods are required for diagnosing early stage cancers.

Single-molecule optical detection has increasingly become an attractive and competitive tool for analytical epigenetics in view of its extreme sensitivity and inherent multiplexing, as well as its potential utility for cost-effective diagnostic applications. Ultra-sensitive single-molecule epigenetic imaging for quantifying and identifying interactions between 5hmC and 5mC have been previously described. See Song, et al., Simultaneous single-molecule epigenetic imaging of DNA methylation and hydroxymethylation, PNAS 113(16): 4338-43 (2016); US 20170298422. However, current methods of single-molecule epigenetic imaging are still blind to the specific genomic location of epigenetic modifications, which information provides additional insight to the diagnosing practitioner.

A need exists for improved methods of single-molecule imaging and localization of epigenetic modifications.

### SUMMARY

Accordingly, described herein is a method for ultra-sensitive optical detection-based single-molecule epigenetic localization (SMEL) for providing loci-specific and strand-specific detection of DNA epigenetic modifications.

In one embodiment, a method for localizing epigenetic modifications of DNA is provided, the method comprising: providing a target DNA strand comprising a least one epigenetic modification, wherein the target DNA strand is annealed to a non-target DNA strand, wherein each of the target DNA strand and the non-target DNA strand is labeled with a first fluorophore at a 3′ end; labeling the at least one epigenetic modification with a second fluorophore; annealing a first probe to the target DNA strand and annealing a second probe to the non-target DNA strand; immobilizing the target DNA strand on a support; and detecting the first and second fluorophores immobilized on the support to localize the at least one epigenetic modification. In embodiments, the method further comprises the step of incubating the reaction product resulting from the annealing of the first and second probes with the target and non-target DNA strands with an exonuclease to digest non-annealed single stranded DNA prior to immobilizing on the support.

In another embodiment, a method of diagnosing cancer in a subject suspected of having cancer is provided, the method comprising: providing a biological sample from the subject, the sample comprising a target DNA strand comprising a least one epigenetic modification, wherein the target DNA strand is annealed to a non-target DNA strand; labeling the target DNA strand and the non-target DNA strand with a first fluorophore at a 3′ end; annealing a first probe to the target DNA strand and annealing a second probe to the non-target DNA strand; immobilizing the target DNA strand on a support; detecting the first and second fluorophores immobilized on the support, wherein detecting comprises imaging via prism-based single molecule total internal reflection fluorescence (TIRF) microscopy, wherein the imaging provides loci-specific and strand-specific localization of the at least one epigenetic modification; comparing the loci-specific and strand-specific localization to a reference epigenetic profile for cancer; and diagnosing the subject as having cancer when the loci-specific and strand-specific localization of the at least one epigenetic modification correlates with the reference epigenetic profile for cancer. In embodiments, the method further comprises the step of incubating the reaction product resulting from the annealing of the first and second probes with the target and non-target DNA strands with an exonuclease to digest non-annealed single stranded DNA prior to immobilizing on the support.

These and other objects, features, embodiments, and advantages will become apparent to those of ordinary skill in the art from a reading of the following detailed description and the appended claims.

### BRIEF DESCRIPTION OF THE FIGURES

FIG. 1A depicts an embodiment of a method of single-molecule epigenetic localization (SMEL) of 5hmC. Target DNA strand (TS) annealed with non-target strand (NTS) are 3′ end labeled with Cy3, and 5hmC is labeled with Cy5. Single strand DNA probe (SP) is 3′ end labeled with biotin and anneals to the TS. Complementary single-strand DNA probe (CSP) anneals to the NTS. The annealed dsDNA can be immobilized to the microscope slide and imaged with single-molecule total internal reflection fluorescence (TIRF) microscopy.

FIG. 1B depicts single-molecule imaging results demonstrate that only the immobilized TS shows Cy5 (5hmC) signal.

FIG. 1C depicts representative images of Cy5 signal for NTS and TS shown in FIG. 1B.

FIG. 1D depicts annealing and immobilization efficiency of different ratios of SP and TS.

FIG. 1E shows 5hmC signal of TS can still be detected among 10¹⁰ non-target dsDNA fragments (NTF).

FIG. 1F shows the detection limit of this method is around 1 pM for TS. 0 pM was in the absence of TS, while imaging buffer was added for movie recording. All error bars represent S.E.M. P-values: ****p<0.0001 by two-tailed Student's t-test.

FIG. 2A depicts a schematic of an embodiment of a purification method to improve detection limit.

FIG. 2B shows that before purification, SP ssDNA competes with TS and occupies most of the neutravidin positions responsible for immobilization. A 5hmC signal image of 1 pM TS before purification is shown at the bottom panel.

FIG. 2C shows that after purification, the SP ssDNA are digested and ultra-pure TS dsDNA are recovered for single-molecule imaging. Purification improves the detection limit to an attomolar level. A 5hmC signal image after purification is shown at the bottom panel.

FIG. 2D shows that after purification, attomolar level of samples can be detected. 0 pM was in the absence of TS, while imaging buffer was added for movie recording. All error bars represent S.E.M. P-values: ****p<0.0001 by two-tailed Student's t-test.

FIG. 3A depicts an embodiment of a method of detection of 5hmC from mESC genomic DNA and human cfDNA by SMEL. Schematic of single-molecule localization of 5hmC epigenetic modification in mESC gDNA and human cfDNA.

FIG. 3B depicts example photo-bleaching traces of single and multiple Cy5 fluorophores, representing one or multiple 5hmC modifications within a single DNA sequence of mESC genome.

FIG. 3C depicts Circle graphs of Cy5 spots (5hmC modification) related to SP3-4, SP5-6, and SP7-8 probes for gDNA.

FIG. 3D depicts circle graphs of Cy5 spots (5hmC modification) related to SP-a and SP-b probes for cfDNA.

FIG. 4 depicts an absorbance spectrum of labeled DNA. DNA with one 5hmC modification was 3′ end labeled with Cy3 and 5hmC labeled with Cy5. Concentrations are calculated using the extinction coefficients of DNA, Cy3, and Cy5.

FIG. 5A shows that before annealing, Cy5 cannot be observed in the presence SP and TS, regardless of the order that they are added.

FIG. 5B illustrates number of Cy3 spots.

FIG. 5C depicts example Cy3 channel images for total DNA showing that both TS and NTS can be immobilized by SP and CSP, respectively.

FIG. 5D depicts FRET histograms show high FRET for TS (right panel), but no FRET for NTS (left panel).

FIG. 5E depicts a representative single molecule time trace showing that every Cy5 signal comes from one single fluorophore.

FIG. 6A is a bar graph of Cy3 spot number from 1 pM TS before purification or 100 aM TS after purification. 0 pM was in the absence of TS.

FIG. 6B depicts representative images of Cy3 signal (total DNA) shown in FIG. 6A. All error bars represent S.E.M. P-values: ****p<0.0001 by two-tailed Student's t-test.

FIG. 7A depicts a schematic of single-molecule localization of 5hmC epigenetic modification in gDNA from mESC.

FIG. 7B illustrates that there is no Cy3 or Cy5 signal for SP1-2 only, gDNA only and SP1-2 and non-TS DNAs. Non-TS DNA does not match to SP1-2, but end-labeled with Cy3 and 5hmC-site labeled with Cy5. For SP1-2 and gDNA, only Cy3 (total DNA) can be detected. For SP3-8, both Cy3 and Cy5 signals can be observed.

FIG. 7C depicts representative Cy3 (total DNA) and Cy5 (5hmC) images for SP1-2 and SP3-8.

FIG. 7D shows Cy5 spots of mESC DNA fragments for SP1-2 and SP3-8 before and after purification. Purification process improves the detection limit. All error bars represent S.E.M. P-values: ****p<0.0001 by two-tailed Student's t-test.

FIG. 8A depicts Cy3 spots of mESC DNA fragments for different SP. A similar level of Cy3 spots for total DNAs is shown.

FIG. 8B depicts Cy5 spots of mESC DNA fragments for different SP. Cy5 spots corresponding to 5hmC cannot be detected for SP1 or SP2.

FIG. 9A depicts example photobleaching traces of one and two Cy5 fluorophores, representing one and two 5hmC modifications in human cfDNA.

FIG. 9B depicts example Cy5 (5hmC) images of cfDNA related to SP-a and SP-b ssDNA probes, respectively.

FIG. 10 depicts genome browser views showing 5hmC levels within and around the probes region in mouse embryonic stem cells.

FIG. 11 depicts genome browser views showing 5hmC level within and around the probes region in cfDNA from healthy donors.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided herein.

While the following terms are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, a “subject” refers to a mammalian subject. Optionally, a subject is a human or non-human primate. Optionally, the subject is selected from the group consisting of mouse, rat, rabbit, monkey, pig, and human. In a specific embodiment, the subject is a human.

The terms “treat,” “treatment,” and “treating,” as used herein, refer to a method of alleviating or abrogating a disease, disorder, and/or symptoms thereof in a subject.

An “effective amount,” as used herein, refers to an amount of a substance (e.g., a therapeutic compound and/or composition) that elicits a desired biological response. In some embodiments, an effective amount of a substance is an amount that is sufficient, when administered to a subject suffering from or susceptible to a disease, disorder, and/or condition, to treat, diagnose, prevent, and/or delay and/or alleviate one or more symptoms of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a formulation to treat a disease, disorder, and/or condition is the amount that alleviates, ameliorates, relieves, inhibits, prevents, delays onset of; reduces severity of and/or reduces incidence of one or more symptoms or features of the disease, disorder, and/or condition. Furthermore, an effective amount may be administered via a single dose or via multiple doses within a treatment regimen. In some embodiments, individual doses or compositions are considered to contain an effective amount when they contain an amount effective as a dose in the context of a treatment regimen. Those of ordinary skill in the art will appreciate that a dose or amount may be considered to be effective if it is or has been demonstrated to show statistically significant effectiveness when administered to a population of patients; a particular result need not be achieved in a particular individual patient in order for an amount to be considered to be effective as described herein.

“Epigenetic modification,” as used herein, refers to modifications of the genome that are heritable, but that do not involve alterations of nucleotide sequence. Epigenetic modifications may be associated with gene activity and expression, or may contribute to other phenotypic traits. Various epigenetic modifications are known, including DNA methylation, RNA modification, and histone modification, which alter how a gene is expressed without modifying the underlying nucleotide sequence. The presently disclosed methods are suitable for detection of epigenetic modifications comprising, for example, methylation of nucleic acids. Epigenetic modifications of DNA detectable by the present methods include, for example, 5-hydroxymethylcytosine (5hmC), 5-methylcytosine (5mC), 5-formylcytosine (5fC), 5-carboxylcytosine (5caC), and the like.

Epigenetic modifications of RNA detectable by the present methods include, for example, as N⁶-methyladenosine (m⁶A).

“Genomic DNA (gDNA),” as used herein, refers to chromosomal DNA that carries biological information of heredity passed from one generation to the next.

“Target DNA strand (TS),” as used herein, refers to a coding DNA strand of interest that comprises at least one epigenetic modification. “Non-target DNA strand (NTS),” as used herein, refers to a noncoding DNA strand that may be annealed to the target DNA strand.

The terms “anneal” and “hybridize” are used interchangeably herein and refer to the phenomena by which complementary nucleic acid strands pair via hydrogen bonding to form a double-stranded polynucleotide. If two nucleic acids are “complementary,” each base of one of the nucleic acids base pairs with corresponding nucleotides in the other nucleic acid. Two nucleic acids need not be perfectly complementary in order to hybridize to one another.

“Biological sample,” as used herein, refers to a clinical sample obtained from a subject for use in the present methods. In embodiments, the biological sample comprises nucleic acids, such as target DNA and/or non-target DNA. In particular embodiments, the biological sample is selected from cells, tissues, bodily fluids, and stool. Bodily fluids of interest include, but are not limited to, blood, serum, plasma, saliva, mucous, phlegm, cerebral spinal fluid, pleural fluid, tears, lactal duct fluid, lymph, sputum, synovial fluid, urine, amniotic fluid, and semen. In a specific embodiment, the biological sample is selected from the group consisting of blood, serum, plasma, urine, tissue, and cultured cells.

“Total internal reflection fluorescence (TIRF) microscopy,” as used herein, refers to a method of microscopy that permits imaging of a thin region of a specimen by exploiting unique properties of an induced evanescent wave or field in a limited specimen region immediately adjacent to the interface between two media having different refractive indices (for example, the contact area between a specimen and a glass coverslip or tissue culture container). Visualization of single-molecule fluorescence with sufficient temporal resolution for dynamic studies is possible with TIRF because of the high signal-to-noise ratio afforded by the evanescent wave excitation.

“Avidin-biotin pairing,” as used herein, refers to an affinity tag pair wherein a first member of the pair is a biotin moiety, and a second member of the pair is selected from the group consisting of avidin, streptavidin, and neutravidin or other modified form of avidin.

As used herein, the term “biotin moiety” refers to an affinity tag that includes biotin or a biotin analogue such as desthiobiotin, oxybiotin, 2-iminobiotin, diaminobiotin, biotin sulfoxide, biocytin, etc. Biotin moieties bind to streptavidin with an affinity of at least 10⁻⁸ M.

As used herein, the term “support” refers to a support (e.g., a planar support such as a microscope slide) that binds biotin or a biotin moiety. In embodiments, the support is linked to avidin, streptavidin, or neutravidin or other modified form of avidin. In a specific embodiment, the support is a polymer-coated quartz surface.

“Localizing” and “localization,” as used herein, refer to determining the location of an epigenetic modification on a target DNA strand. In embodiments, the disclosed methods permit strand-specific and/or loci-specific localization of discrete epigenetic modifications of genomic and cf DNA, such as 5hmC, 5mC, and the like.

Disclosed herein is a method of single-molecule epigenetic localization (SMEL), a single-molecule optical detection-based method for loci-specific and strand-specific epigenetic modification imaging. SMEL achieves attomolar ultrasensitivity and is applied herein to image genomic DNA and cfDNA to demonstrate its utility and clinical application.

In one embodiment, a method for localizing epigenetic modifications of DNA is provided, the method comprising: (a) providing a target DNA strand comprising a least one epigenetic modification, wherein the target DNA strand is annealed to a non-target DNA strand, wherein each of the target DNA strand and the non-target DNA strand is labeled with a first fluorophore at a 3′ end; (b) labeling the at least one epigenetic modification with a second fluorophore; (c) annealing a first probe to the target DNA strand and annealing a second probe to the non-target DNA strand; (d) immobilizing the target DNA strand on a support; and (e) detecting the first and second fluorophores immobilized on the support to localize the at least one epigenetic modification.

DNA strands may include genomic DNA and/or cfDNA from a eukaryotic source, including, but not limited to, plants, animals (e.g., reptiles, mammals, insects, worms, fish, etc.), fungi (e.g., yeast), and the like, as well as genomic DNA isolated from tissue samples. In certain embodiments, the DNA used in the disclosed method is derived from a biological sample obtained from mammal, such as a human.

In some embodiments, the biological sample is obtained from a subject that has or is suspected of having a disease or condition associated with epigenetic modifications, such as a cancer, inflammatory disease, or pregnancy. In some embodiments, the biological sample may be made by extracting fragmented DNA from a fresh or archived patient sample, e.g., a formalin-fixed paraffin embedded tissue sample. In other embodiments, the biological sample may be a sample of cfDNA from a bodily fluid, e.g., peripheral blood.

The DNA used in the initial steps of the method comprises non-amplified DNA and, in certain embodiments, has not been denatured beforehand.

In embodiments, the DNA is fragmented for use in the instant methods. DNA may be fragmented mechanically (e.g., by sonication, nebulization, or shearing) or enzymatically, using a double-stranded DNA fragmentase enzyme (New England Biolabs, Ipswich Mass.). In other embodiments, the DNA in the initial sample may already be fragmented (e.g., as is the case for FFPE samples and cfDNA, e.g., ctDNA (circulating tumor DNA)).

In some embodiments, the fragments in the initial sample may have a median size that is below 1 kb (e.g., in the range of 50 bp to 500 bp, 80 bp to 400 bp, or 100-1,000 bp), although fragments having a median size outside of this range may be used. Cell-free or circulating tumor DNA (ctDNA), i.e., tumor DNA circulating freely in the blood of a cancer patient, is highly fragmented, with a mean fragment size about 165-250 bp. cfDNA can be obtained by centrifuging whole blood to remove all cells, and then analyzing the remaining plasma.

First and second fluorophores are optically-distinguishable, such that moieties labeled with first and second fluorophores can be independently detected. Various fluorophore pairs are known in the art and suitable for use in the present methods. Suitable distinguishable fluorescent label pairs for use in the disclosed methods include, but are not limited to, Cy-3 and Cy-5 (Amersham Inc., Piscataway, N.J.). Quasar 570 and Quasar 670 (Bioseareh Technology, Novato, Calif.), Alexa Fluor 555 and Alexa Fluor 647 (Molecular Probes, Eugene, Oreg.), BODIPY V-1002 and BODIPY V-1005 (Molecular Probes, Eugene, Oreg.), POPO-3 and TOTO-3 (Molecular Probes, Eugene, Oreg.), PO-PRO3 TO-PRO3 (Molecular Probes. Eugene. Oreg.), and the like. Further suitable distinguishable detectable labels may be found in Kricka, Stains, labels and detection strategies for nucleic acid assays, Ann. Clin. Biochem. 39(2): 114-29, (2002).

Each of the target DNA strand and the non-target DNA strand are end-labeled at a 3′ end with a first fluorophore. Methods of end-labeling DNA are known in the art, and include, for example, terminal transferase reactions.

In embodiments, the at least one epigenetic modification is selected from the group consisting of 5-hydroxymethylcytosine (5hmC), 5-methylcytosine (5mC), 5-formylcytosine (5fC), and 5-carboxylcytosine (5caC). In a specific embodiment, the at least one epigenetic modification comprises 5hmC.

5hmC epigenetic modifications in target DNA strands are labeled by incubating target DNA with a DNA β-glucosyltransferase and UDP glucose modified with a chemoselective group, thereby covalently labeling the hydroxymethylated DNA molecules with the chemoselective group, and linking the first fluorophore to the chemoselectively-modified DNA via a cycloaddition reaction. The hydroxymethylated DNA molecules in the target DNA strand are labeled with a with a chemoselective group that can participate in a click reaction. This step may be accomplished by incubating the adaptor-ligated cfDNA with DNA β-glucosyltransferase (e.g., T4 DNA β-glucosyltransferase (commercially available from a number of vendors, although other DNA β-glucosyltransferases exist) and, e.g., UDP-6-N₃-Glu (i.e., UDP glucose containing an azide). This step may be done using a protocol adapted from US20110301045 or Song et al, Selective chemical labeling reveals the genome-wide distribution of 5-hydroxymethylcytosine, Nat. Biotechnol. 29(1): 68-72 (2011), for example.

The present methods utilize probe pairs, wherein a first single-stranded DNA probe is designed to be complementary to the target DNA strand, and the second single-stranded DNA probe is designed to be complementary to the non-target DNA strand. In embodiments, the first and second probes are complementary to each other.

The ratio of first probe to target DNA strand is selected to provide an excess of probe, in order to facilitate capture of as much target DNA as possible. In embodiments, the ratio of first probe to target DNA strand is about 10:1, about 100:1, about 1000:1, or about 10,000:1. In a specific embodiment, the ratio of first probe to target DNA is about 100:1.

First and second probes are labeled with a biotin moiety to enable capture on a suitable support, which is correspondingly labeled with a surface-tethered moiety that binds a biotin moiety. In embodiments, the first and second probes are labeled with biotin and the support comprises a surface-tethered moiety selected from the group consisting of avidin, streptavidin, and neutravidin. In this way, target DNA strands may be captured and immobilized on a support via avidin-biotin pairing.

To prepare DNA fragments for single-molecule imaging, labeled DNA fragments are mixed with corresponding single-stranded DNA probes in different molar ratios under annealing conditions. For example, labeled DNA fragments are mixed with corresponding single-stranded DNA probes in annealing buffer, heated to denature the DNA fragments, and then cooled to facilitate annealing of first and second probes to each of the target and non-target DNA strands, respectively. The newly annealed DNA (fluorophore-labeled and conjugated with a biotin moiety) is then ready for immobilization and imaging.

Optionally, prior to immobilizing and imaging, the product resulting from the annealing step described above may be further purified to improve the detection level of the assay. Specifically, the product of the annealing step is optionally incubated with an exonuclease to digest excess single-stranded DNA. In a specific embodiment, the product of the annealing step is incubated with E. coli Exonuclease I to digest single-stranded DNA. Advantageously, including this purification step to remove excess single-stranded DNA enhances the detection limit of SMEL to attomolar levels.

Immobilizing labeled DNA molecules on a support, such as a microscope slide, is accomplished using a slide coated in a binding partner for the capture tag added to the DNA molecules. For example, in some embodiments, DNA molecules labeled with a biotin moiety may be captured on a slide coated in avidin, streptavidin, or neutravidin. These slides may be made by first passivating the slides in a mixture of polyethylene glycol (PEG) mPEG-SVA and biotin-PEG-SVA (at a ratio of, e.g., 99:1 (mol/mol)) to reduce non-specific binding of the DNA, and then coating the slide in avidin, streptavidin, or neutravidin. The labeled DNA molecules can be immobilized on the surface of the slide, e.g., at a concentration of 10-300 pM (e.g., 30-100 pM) for a period of time, e.g., 5 minutes to 1 hour, e.g., 15 minutes. The support is washed to remove unbound DNA.

Individual molecules of epigenetically modified DNA are imaged on the support at a single-molecule resolution. Imaging may employ any sensitive, high resolution, fluorescence detector equipped to excite each of the first and second fluorophores. Appropriate filters should be used so that the signals from the first and second fluorophores can be separately detected and imaged. In one embodiment, the imaging employs total internal reflection fluorescence (TIRF) microscopy. For TIRF microscopy, a dual-laser excitation system is used to excite each of the first and second fluorophores. Total fluorescence signals from first and second fluorophores are collected by a water immersion objective lens and passed through a notch filter to block excitation beams. Emission signals from the second fluorophore (i.e., labeled epigenetic modification(s)) are separated by a dichroic mirror and detected by an electron-multiplying charge-coupled device camera. Data are recorded to provide fluorescence intensity signal and/or time trajectories of individual molecules.

After the labeled DNA molecules have been imaged, the method may further comprise counting the number of individual molecules labeled with the first and second fluorophores, thereby determining the number of epigenetically modified DNA molecules in the sample.

Imaging provides loci-specific and/or strand-specific localization of at least one epigenetic modification of the DNA.

The method described above may be generally applied to analyze biological DNA samples. For example, in some embodiments, the method in a method that involves: (a) localizing, using the method described above: (i) epigenetic modifications in a first sample of DNA and (ii) epigenetic modifications in a second sample of DNA; and (b) comparing the results obtained in step (a) to determine if there is a difference in epigenetic profile between the samples. At least one of the samples is a clinical sample, a sample containing DNA obtained from a patient.

“Epigenetic profile,” as used herein, refers to a loci-specific and strand-specific epigenetic modification signature determined by the instant methods for a given DNA sample. In embodiments, the “reference epigenetic profile” for cancer or for a particular type of cancer is determined by carrying out the disclosed methods on one or more control samples. Loci- and strand-specific epigenetic modification data is collected from the reference population to provide a reference epigenetic profile. In embodiments, the control is an external control, such that imaging data obtained from the subject to be diagnosed is compared to imaging data from individuals known to suffer from, or known to be at risk of, a given condition (i.e., the reference population). In other embodiments, the imaging data obtained from the subject to be diagnosed is compared to imaging data from normal, healthy individuals. It should be understood that the reference population may consist of approximately 20, 30, 50, 200, 500 or 1000 individuals, or any value therebetween.

In some embodiment, the different samples may consist of an “experimental” sample. i.e., a sample of interest, and a “control” sample to which the experimental sample may be compared. In embodiments, the different samples are pairs of cell types or fractions thereof, one cell type being a cell type of interest, e.g., an abnormal cell, and the other a control, e.g., normal, cell. If two fractions of cells are compared, the fractions are usually the same fraction from each of the two cells. In certain embodiments, however, two fractions of the same cell may be compared. Exemplary cell type pairs include, for example, cells isolated from a tissue biopsy (e.g., from a tissue having a disease such as colon, breast, prostate, lung, skin cancer, or infected with a pathogen etc.) and normal cells from the same tissue, usually from the same patient; cells grown in tissue culture that are immortal (e.g., cells with a proliferative mutation or an immortalizing transgene), infected with a pathogen, or treated (e.g., with environmental or chemical agents such as peptides, hormones, altered temperature, growth condition, physical stress, cellular transformation, etc.), and a normal cell (e.g., a cell that is otherwise identical to the experimental cell except that it is not immortal, infected, or treated, etc.); a cell isolated from a mammal with a cancer, a disease, a geriatric mammal, or a mammal exposed to a condition, and a cell from a mammal of the same species, preferably from the same family, that is healthy or young; and differentiated cells and non-differentiated cells from the same mammal (e.g., one cell being the progenitor of the other in a mammal, for example). In one embodiment, cells of different types. e.g., neuronal and non-neuronal cells, or cells of different status (e.g., before and after a stimulus on the cells) may be employed. In another embodiment of the invention, the experimental material is cells susceptible to infection by a pathogen such as a virus, e.g., human immunodeficiency virus (HIV), etc., and the control material is cells resistant to infection by the pathogen. In another embodiment of the invention, the sample pair is represented by undifferentiated cells, e.g., stem cells, and differentiated cells.

The methods described above may be used to identify an epigenetic modification signature, or profile, that correlates with phenotype. e.g., a disease, condition or clinical outcome, etc. In some embodiments, this method may comprise (a) performing the above-described method on a plurality of DNA samples, wherein the DNA samples are isolated from patients having a known phenotype, e.g., disease, condition or clinical outcome, thereby determining a signature of epigenetic modification in DNA from each of the patients; and (b) identifying an epigenetic profile that is correlated with the phenotype.

In some embodiments, the epigenetic profile may be diagnostic (e.g., may provide a diagnosis of a disease or condition or the type or stage of a disease or condition, etc.), prognostic (e.g., indicating a clinical outcome, e.g., survival or death within a time frame), or theranostic (e.g., indicating which treatment would be the most effective).

Also provided is a method for analyzing a patient sample. In this embodiment, the method may comprise: (a) identifying, using the above-described method, an epigenetic profile in the DNA of a patient; (b) comparing the identified sequences to a reference epigenetic profile that correlates with a phenotype, e.g., a disease, condition, or clinical outcome etc.; and (c) providing a report indicating a correlation with phenotype. This embodiment may further comprise making a diagnosis, prognosis or theranosis based on the results of the comparison. It should be understood that the present methods are applicable to a wide range of diseases, conditions, or clinical outcomes characterized by epigenetic modifications to nucleic acids.

In a specific embodiment, the method comprises (a) providing a biological sample obtained from the subject suspected of having cancer comprising a target DNA strand comprising at least one epigenetic modification, wherein the target DNA strand is annealed to a non-target DNA strand; (b) labeling the target DNA strand and the non-target DNA strand with a first fluorophore at a 3′ end; (c) annealing a first probe to the target DNA strand and annealing a second probe to the non-target DNA strand; (d) immobilizing the target DNA strand on a support; (e) detecting the first and second fluorophores immobilized on the support, wherein detecting comprises imaging via prism-based single molecule total internal reflection fluorescence (TIRF) microscopy, wherein the imaging provides loci-specific and strand-specific localization of at least one epigenetic modification; (f) comparing the loci-specific and strand-specific localization to a reference epigenetic profile for cancer; and (g) diagnosing the subject as having cancer when the loci-specific and strand-specific localization of step (e) correlates with the reference epigenetic profile for cancer. Optionally, the method comprises purifying the product of the annealing step (c) by digesting with an exonuclease prior to immobilizing target DNA.

In embodiments, the subject is diagnosed with cancer when the subject's epigenetic profile is concordant with the reference epigenetic profile for cancer. In a specific embodiment, the subject is diagnosed with cancer when the subject's epigenetic profile is at least 80% concordant with the reference epigenetic profile.

“Concordant,” as used herein, refers to the degree of identity between compared datasets, including imaging, or epigenetic profile, datasets. In certain embodiments, concordant refers to at least 25%, at least 50%, at least 75%, at least 80%, at least 85%, at least 90%, at least 91%, at least 92%, at least 93%, at least 94%, at least 95%, at least 96%, at least 98%, at least 99%, or 100% identity.

In embodiments, the method further comprises treating the diagnosed patient with an effective amount of a therapeutic agent specific for the cancer diagnosed.

While cancer is an exemplary disease for application of the instant methods, it should be understood that the disclosed methods may be applied to any disease, condition, or clinical outcome characterized by epigenetic modifications to nucleic acids. Such diseases, conditions, or clinical outcomes may be assessed via SMEL, using single-stranded probes designed to be complementary to known genomic regions having epigenetic modifications associated with said disease, condition, or clinical outcome.

In other embodiments, the presently disclosed methods are suitable for use in identifying epigenetic patterns or profiles of DNA from other species, including plant and animal species. For example, single-stranded probes designed to be complementary to known genomic regions having epigenetic modifications can be employed in the instant methods to rapidly determine a source of DNA.

### EXAMPLES

The following examples are given by way of illustration and are in no way intended to limit the scope of the present disclosure.

### Example 1. Materials and Methods

mESCs Culture and Preparation of Genomic DNA

Mouse embryonic stem cells (mESCs) E14 were cultured on gelatin-coated plates in Dulbecco's Modified Eagle Medium (DMEM) supplemented with 15% FBS, 2 mM L-glutamine, 1× non-essential amino acids, 1× penicillin/streptavidin, 0.1 mM β-mercaptoethanol, 10 ng/ml leukemia inhibitory factor (LIF), 1 μM PD0325901, and 3 μM CHIR99021.

Genomic DNA was extracted with Quick-DNA Plus kit (Zymo Research) following manufacturer's protocol. Genomic DNA was fragmented with dsDNA Fragmentase (NEB) and size selected with AMPure XP beads (Beckman Coulter) to 50-200 bp fragments.

Labeling of Synthetic DNA and mESC Genomic DNA

Synthetic DNA or genomic DNA fragments were end-labeled with Cy3 by incubating 1 μg DNA with 1× Terminal Transferase Reaction Buffer (NEB), 0.25 mM CoCl₂, 0.2 mM Cy3-dCTP (GE Healthcare), and 40 U Terminal Transferase (NEB) in a 20-μl solution for 2 hours at 37° C. 40 U Terminal Transferase (NEB), Terminal Transferase Reaction Buffer (NEB), CoCl₂ and H₂O were added to make a 30-μl solution and incubated for 2 hours at 37° C. The end-labeled DNA was purified with Oligo Clean & Concentrator (Zymo Research) and eluted in 10 μl H₂O. Cy3 end-labeled DNA was incubated with 50 mM HEPES buffer (pH 8.0), 25 mM MgCl₂, 150 μM UDP-6-azide-glucose (Jena Bioscience), and 10 U T4 β-glucosyltransferase (Thermo Scientific) in a 20-μl solution for 1 h at 37° C. 5 μl Cy5 DBCO (10 mM stock in DMSO; Sigma) was directly added to the reaction mixture and incubated for 24 hours at 37° C. The labeled DNA was purified with Oligo Clean & Concentrator (Zymo Research) and eluted in 10 μl low-EDTA TE buffer.

### Labeling of Cell-Free DNA

Cell-free DNA (cfDNA) was end-labeled with Cy3 by incubating 20 ng cfDNA with 1× Terminal Transferase Reaction Buffer (NEB), 0.25 mM CoCl₂, 0.1 mM Cy3-dCTP (GE Healthcare), and 20 U Terminal Transferase (NEB) in a 10-μl solution for 40 min at 37° C. The end-labeled DNA was purified with Oligo Clean & Concentrator (Zymo Research) and eluted in 8.5 μl H₂O. Cy3 end-labeled DNA was incubated with 50 mM HEPES buffer (pH 8.0), 25 mM MgCl₂, 150 μM UDP-6-azide-glucose (Jena Bioscience), and 5 U T4 β-glucosyltransferase (Thermo Scientific) in a 10-μl solution for 1 h at 37° C. Then 2.4 μl Cy5 DBCO (10 mM stock in DMSO; Sigma) was directly added to the reaction mixture and incubated for 24 hours at 37° C. The labeled DNA was purified with Oligo Clean & Concentrator (Zymo Research) and eluted in 7 μl low-EDTA TE buffer.

### Preparation of DNA Fragments for Single-Molecule Imaging

All of the single-stranded DNA probes with biotin at the 3′ end were obtained from Integrated DNA Technologies (IDT). To prepared DNA fragments for single-molecule imaging, labeled DNA fragments with or without 5hmC were mixed with corresponded single-stranded DNA probes in different molar ratio in annealing buffer (10 mM Tris, 1 mM EDTA, 50 mM NaCl, pH 8.0), heated for 3 min and slow cooled to room temperature for ˜2 h. The new annealed DNA (dye-labeled and conjugated with biotin) was ready for single-molecule imaging.

For purification assays, annealed DNA was further digested by E. coli Exonuclease I (NEB) in reaction buffer (67 mM Glycine-KOH, 6.7 mM MgCl₂, 10 mM 2-mercaptoethanol, pH 9.5) for 1.5 h at 37° C., to remove excess and nonspecific single-stranded DNA with biotin. Next, heat inactivation of the Exonuclease I was performed by 20 min incubating at 80° C. Finally, the annealed DNA was purified with Oligo Clean & Concentrator (Zymo Research) and eluted in 15 μl T50 (10 mM Tris-HCl pH 8.0, 50 mM NaCl).

### Single-Molecule Imaging

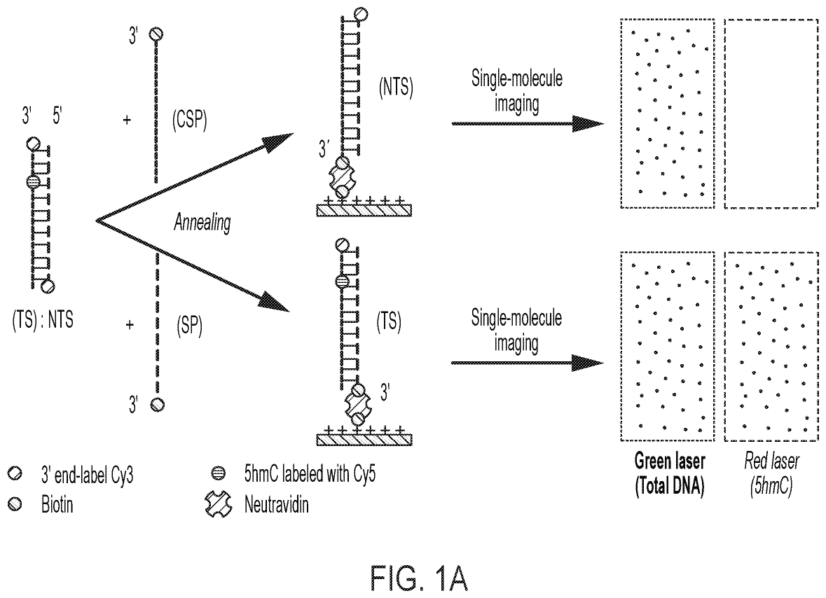

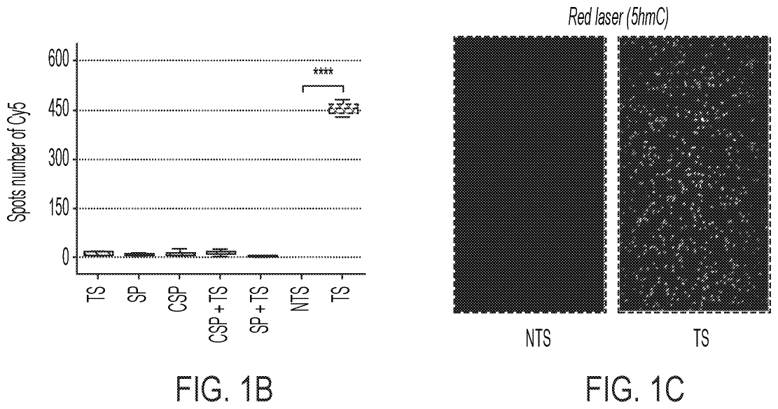

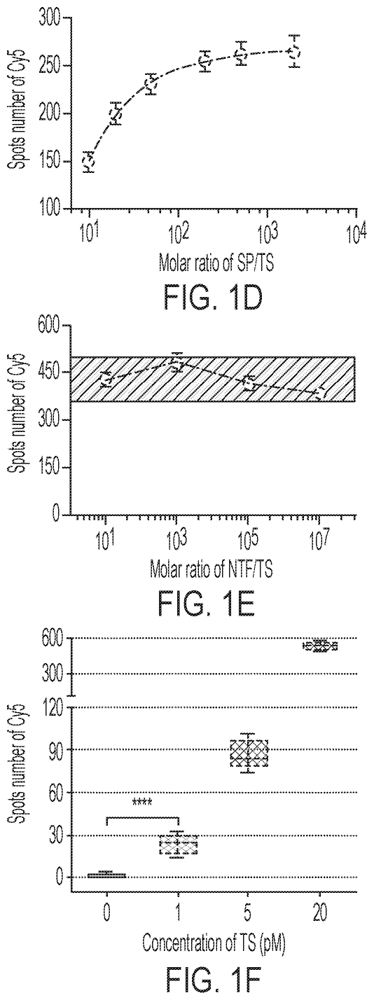

To immobilize DNA sample for SMEL detection, a quartz slide was first coated with a mixture of 97% mPEG (Laysan Bio) and 3% biotin PEG (Laysan Bio), and then flow chambers were assembled using strips of double-sided tape and epoxy. 0.05 mg/ml neutravidin solution was flowed into each flow chamber and incubated for 5 min. The dye-labeled DNAs conjugated with biotin were injected into the chamber, and then were immobilized on the PEG-coated surface via biotin-neutravidin interaction by 15 min incubation, as shown in FIG. 1A. After washing out the free DNAs, subsequent single-molecule imaging was performed in imaging buffer, containing an oxygen scavenging system consisting of 0.8 mg/ml glucose oxidase, 0.625% glucose, 3 mM Trolox and 0.03 mg/ml catalase.

### Data Acquisition and Analysis

Single-molecule imaging was conducted by a prism-type total internal reflection fluorescence (TIRF) microscope. The excitation beam was focused into a pellin broca prism (Altos Photonics), which was placed on top of a quartz slide with a thin layer of immersion oil in between to match the index of refraction. For the TIRF microscope, a dual-laser excitation system (532 and 640 nm Crystal Laser) was equipped to excite Cy3 and Cy5 fluorophores. The fluorescence signals from Cy3 and Cy5 were collected by a water immersion objective lens (60×, 1.2 N.A. Nikon) and then passed through a notch filter to block out excitation beams. The emission signals of Cy5 dyes were separated by a dichroic mirror (FF662-FDi01; Semrock) and detected by the electron-multiplying charge-coupled device camera (iXon 897; Andor Technology). Data were recorded with a time resolution of 200 ms as a stream of imaging frames and analyzed with scripts written in interactive data language to give fluorescence intensity signal or time trajectories of individual molecules.

For total DNA signal (Cy3) or 5hmC signal (Cy5), short movies (2 sec) were recorded from 10 to 20 random locations, excited by green laser (532 nm) and red laser (640 nm), respectively. Statistical analysis of spot number was performed automatically using smCamera software. For real-time trajectories of individual DNA molecules with 5hmC, long movies (3 min) were recorded from 5 to 10 random locations to detect photo-bleaching event of Cy5. In order to account for variations between experiments, a calibration control (in the absence of dye-labeled DNA sample) was performed prior to testing, as shown in FIG. 1F (0 μM).

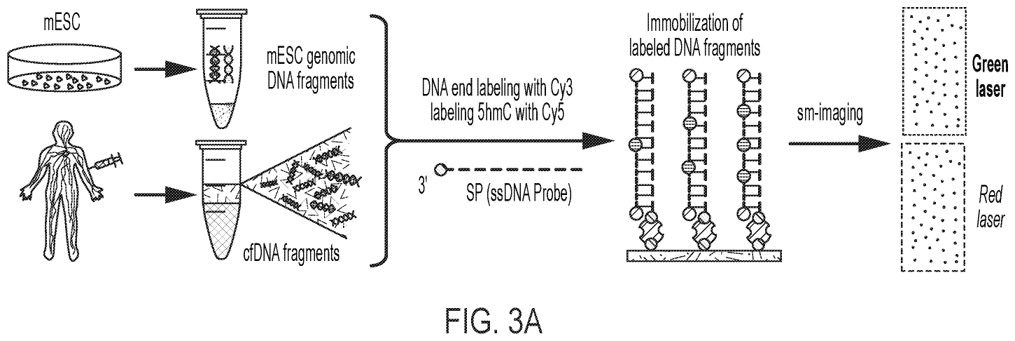

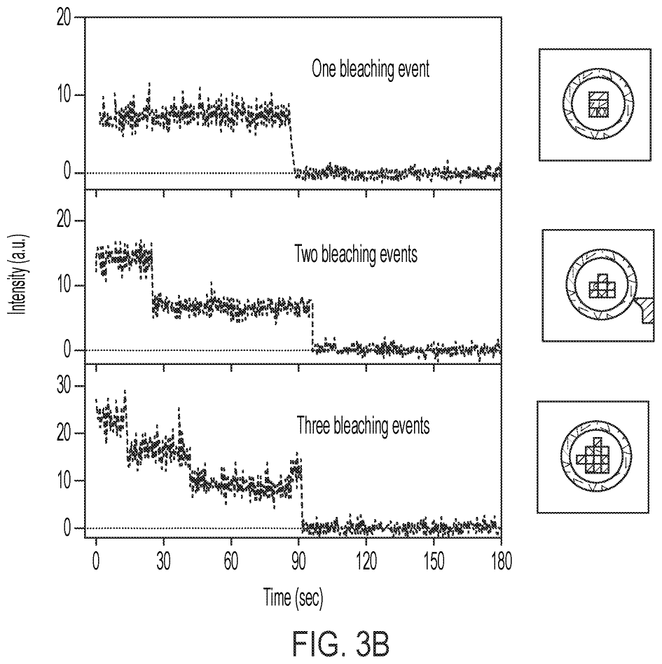

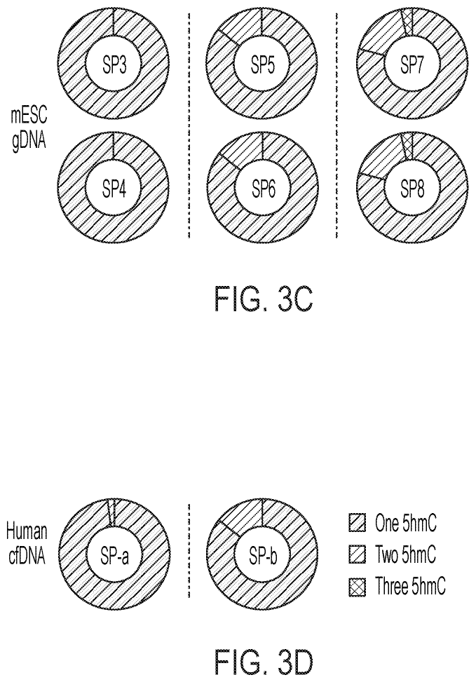

Basic data analysis was carried out by the smCamera software written in C++(Microsoft). Spots number of Cy3/Cy5 was collected from at least ten independent short movies. Traces with Cy5 photo-bleaching were collected from at least five independent long movies. The number of molecules used in FIGS. 3C and 3D are shown in Table 4, below.

### Example 2. Loci-Specific and Strand-Specific Imaging

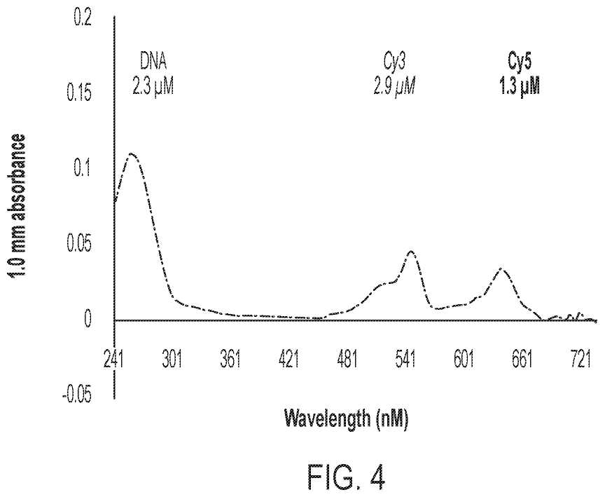

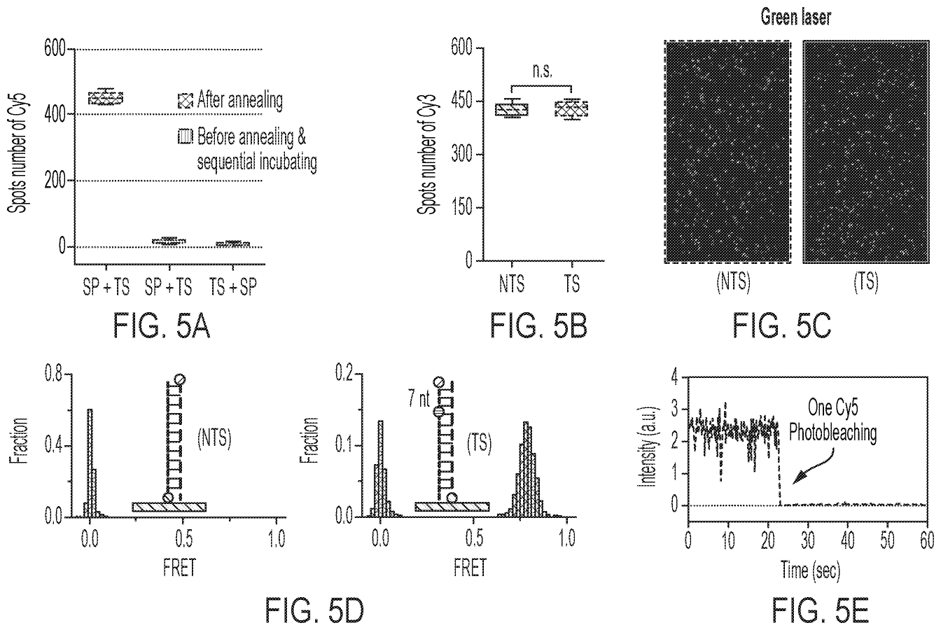

The presently disclosed method combines a selective chemical labeling strategy, single molecule fluorescent imaging technique with a purification system to improve detecting limit. To optically localize 5hmC, each of the target DNA strand (TS) having a 5hmC modification and the annealed non-target DNA strand (NTS) are 3′ end-labeled with Cy3 and 5hmC is labeled with Cy5 (FIG. 4). A single-strand DNA probe (SP) and its complementary single-strand DNA probe (CSP) are designed and labeled with biotin and match to the TS and NTS, respectively (Table 1). In this way, by annealing with the biotin-labeled SP, the dye-labeled TS can be captured via surface-tethered neutravidin on a polymer-coated quartz surface and imaged with a prism-based single-molecule total internal reflection fluorescence (TIRF) microscope. By counting the fluorophores in red channel (Cy5) and green channel (Cy3), the number of 5hmC-containing molecules and the total amount of sequence-specific DNA fragments can be quantified, respectively (FIG. 1A). As expected, only annealed TS DNA showed significant 5hmC (Cy5) signal (FIGS. 1B and 1C and FIG. 5A), while both annealed TS and NTS showed similar total amount of DNA fragments (Cy3) (FIG. 5B). Since 5hmC position (Cy5) was just 7 base pairs away from Cy3 labeled 3′ end, as a double-confirmation, high FRET was detected based on annealed TS but not NTS (FIG. 5C).

The suitable ratio of SP to TS for annealing and the detection limit of this probing strategy were assessed (FIGS. 1D and 1E). The results achieved confirm that the disclosed method is highly efficient and, advantageously, has a high signal-to-noise ratio. In addition, the Cy5 intensity trace demonstrates that each spot in the Cy5 channel represents only one fluorophore using photo-bleaching (FIG. 5D). To determine the detection limit, different concentrations of annealed TS were utilized for single-molecule imaging and the results suggested that the concentration limit of this method is around 1 picomolar (pM) (FIG. 1F). These results demonstrate SMEL is capable of both loci-specific and strand-specific 5hmC imaging.

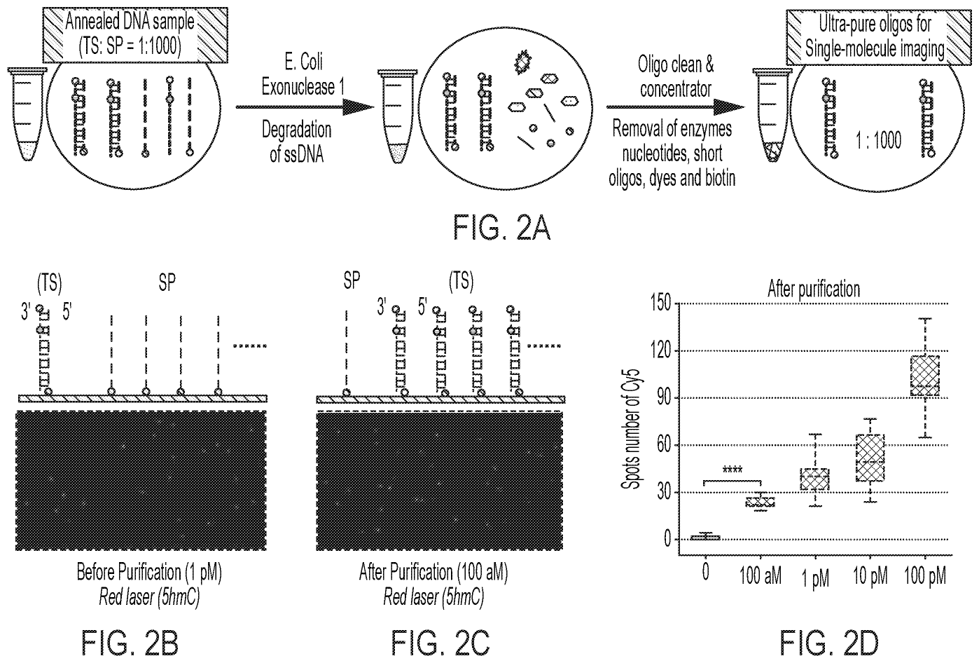

TABLE 1
DNA sequence information used in FIGS. 1 and 2.
Underlined C represents 5hmC

| Name | Sequence (5′-3′) |
|---|---|
| TS (target DNA strand) | CCCGACGCATGATCTGTACTTGATCGACCGTGCAAC-Cy3 |
| (SEQ ID NO: 1) | |
| NTS (non-target DNA | GTTGCACGGTCGATCAAGTACAGATCATGCGTCGGG-Cy3 |
| strand) | |
| (SEQ ID NO: 2) | |
| SP (ssDNA probe) | GTTGCACGGTCGATCAAGTACAGATCATGCGTCGGG-Biotin |
| (SEQ ID NO: 3) | |
| CSP (Complementary | CCCGACGCATGATCTGTACTTGATCGACCGTGCAAC-Biotin |
| ssDNA probe) | |
| (SEQ ID NO: 4) | |

### Example 3. Purification Enhances the Detection Limit of SMEL

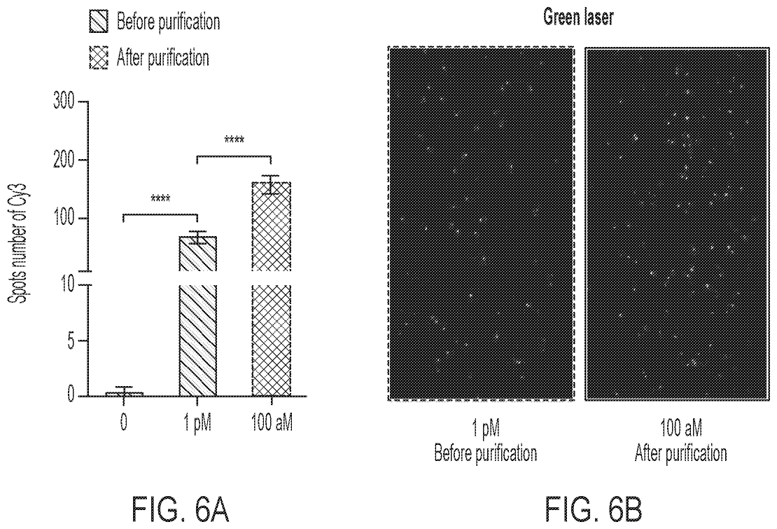

To achieve more sensitive 5hmC modification detection, a purification process is optionally applied to the annealed TS sample. As shown in FIG. 1D, 1000 times more SP than TS is used to anneal and capture as much TS with 5hmC as possible. However, most of the surface-tethered neutravidin would thus be occupied by SP, which is 3′ end-labeled with biotin and can compete with annealed TS (FIG. 2B). To overcome this problem, the annealed TS sample is incubated with E. Coli Exonuclease I to digest single-stranded DNA (ssDNA) in 3′ to 5′ direction (FIG. 2A). This step enables the efficient elimination of excess single strand SP. Advantageously, the added purification step surprisingly improves the detection limit of SMEL by 10,000 fold: from 1 μM to 100 attomolar (aM) (FIGS. 2C-2D; FIGS. 6A-6B).

### Example 4. Application of SMEL to gDNA

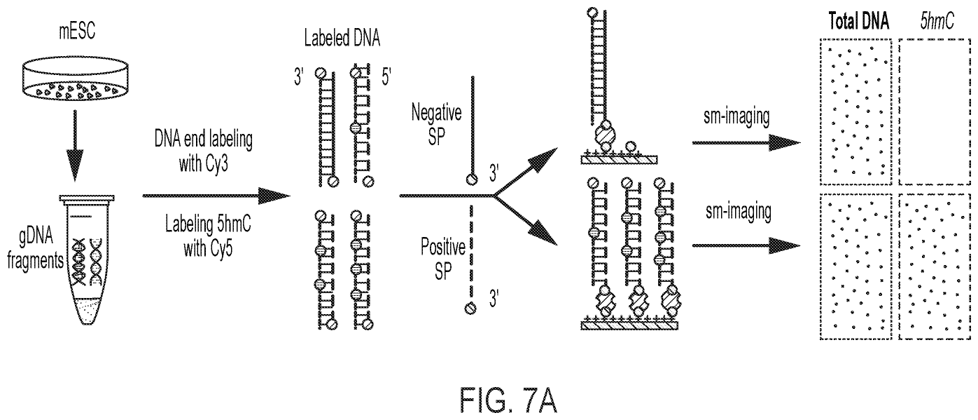

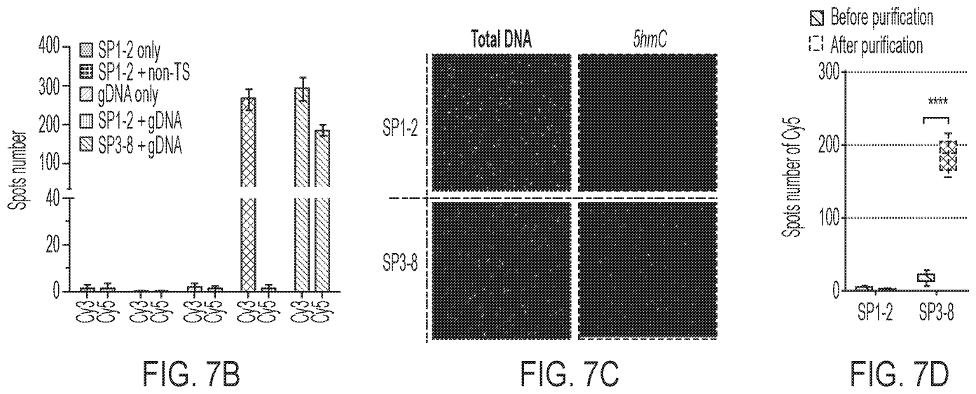

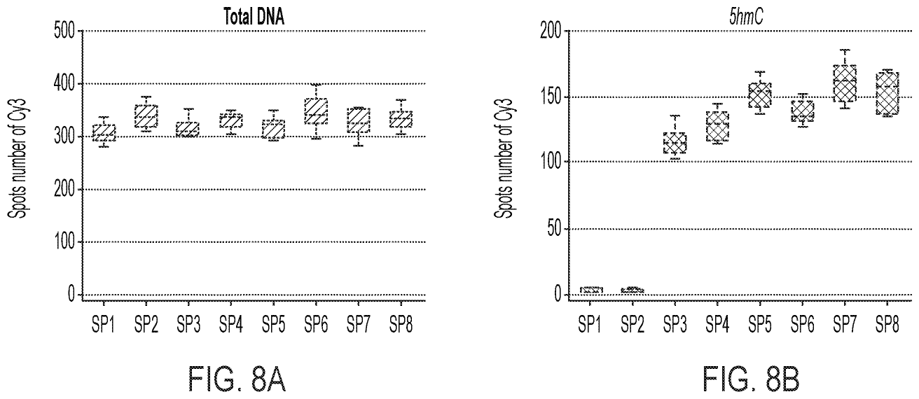

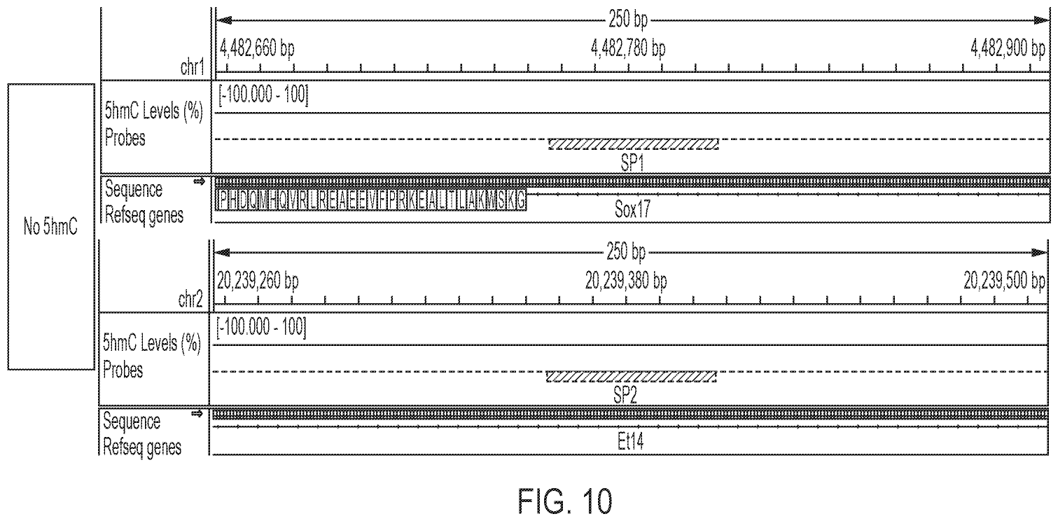

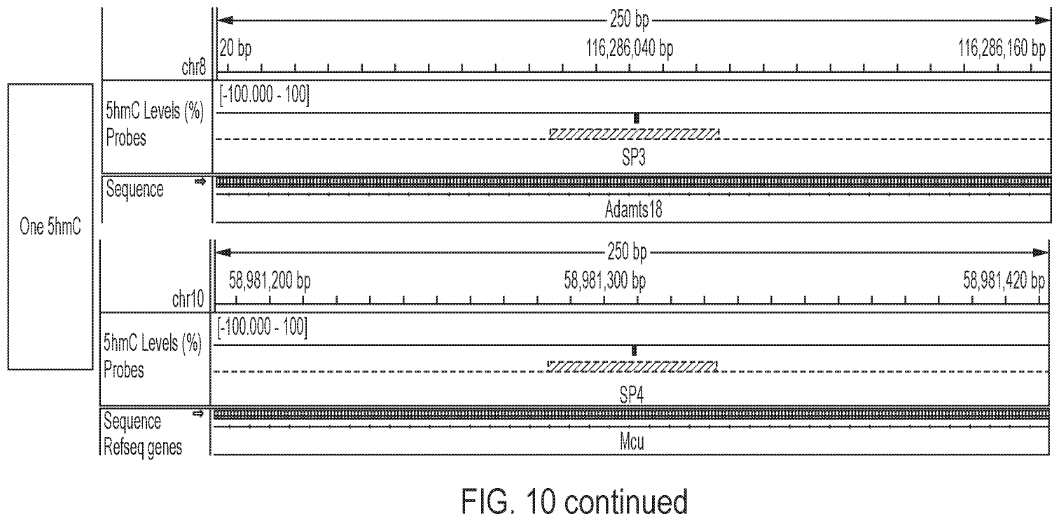

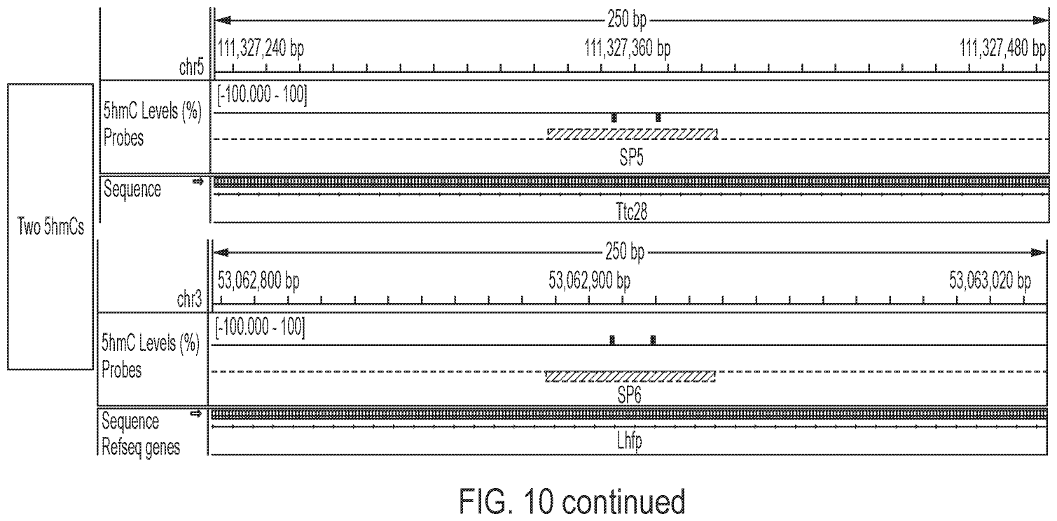

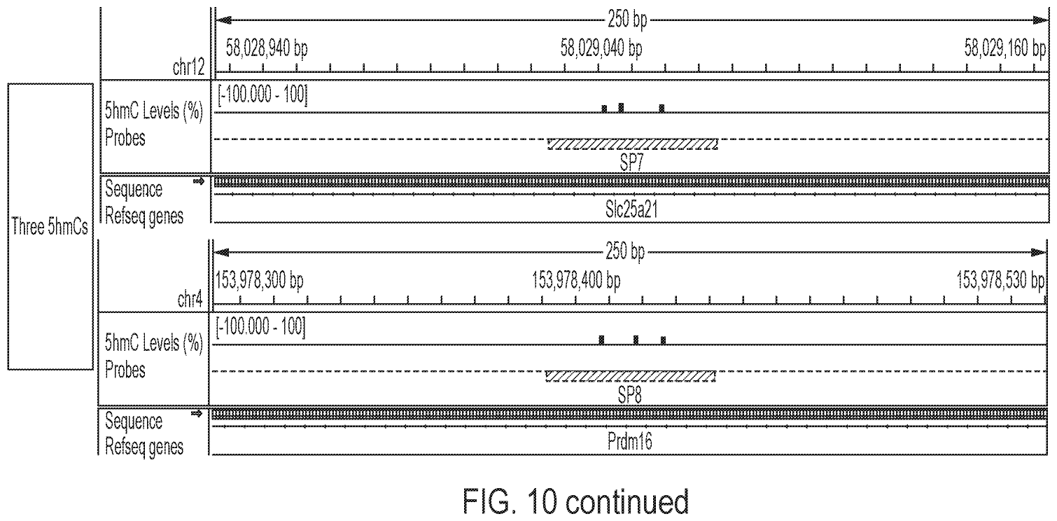

To evaluate the performance of SMEL, its ability to detect known 5hmC sites in real genomic DNA (gDNA) samples from mESC was assessed, gDNA was first extracted from mESC and then fragmented to 50-200 bp for labeling, as described in FIG. 7A. Based on published base-resolution sequencing of 5hmC in mESC, a series of ssDNA probes were designed for single-molecule optical imaging and for single or multiple 5hmC modifications detection (Table 2 and FIG. 10): SP1-2 are negative controls that target sequences that do not contain 5hmC; SP3-4, SP5-6, and SP7-8 target sequences that contain one, two, and three 5hmCs, respectively. As described above, the number of 5hmC and total amount gDNA fragments can be determined by counting the fluorophores in the red and green channels, respectively (FIGS. 7A-7D). As expected, the detection limit of SMEL is significantly improved by the purification system, and SP1-2 probes were not capable for 5hmC quantification (FIGS. 8A-8B). In addition to calculating the fluorophore numbers in individual gDNA fragments, we also checked single or multiple 5hmC modifications based on photo-bleaching events (FIG. 3B), which confirms that three 5hmC modifications were only detected with SP7-8, while no more than one 5hmC was observed with SP3-4 (FIG. 3C). The data demonstrate the high efficiency and ultra-high sensitivity of SMEL. In this way, the location of 5hmC is validated by single-molecule imaging in a loci-specific and strand-specific manner.

TABLE 2
Genomic DNA and corresponding SP (single-stranded probe) sequences

| Name | | Position | Sequence (5′-3′) |
|---|---|---|---|
| No | Sequence 1 | chr1: | GAAAGGTGGAGAGGCGCGCAGGGTTACCCGAG |
| 5hm | (SEQ ID NO: 5) | 4482757-4482806 | TGAGCTCCGGCACCCTGA |
| C | SP1 | | TCAGGGTGCCGGAGCTCACTCGGGTAACCCTG |
| | (SEQ ID NO: 6) | | CGCGCCTCTCCACCTTTC-Biotin |
| | Sequence 2 | chr2: | GAAATGCTTTGCATCCCTCTCGAGCCTGGCCA |
| | (SEQ ID NO: 7) | 20239356-20239405 | TATAGGTAATGGCTTTGC |
| | SP2 | | GCAAAGCCATTACCTATCTGGACAGGCTCGAG |
| | (SEQ ID NO: 8) | | AGGGACGCCAAGCATTTC-Biotin |
| One | Sequence 3 | chr8: | TTATCTTCAAGGCCTTCATTGTGCCGTCATTG |
| 5hm | (SEQ ID NO: 9) | 116286022-116286071 | TTAGCGCTTTCAACCTTT |
| C | SP3 | | AAAGGTTGAAAGCGCTAACAATGACGGCACAA |
| | (SEQ ID NO: 10) | | TGAAGGCCTTGAAGATAA-Biotin |
| | Sequence 4 | chr10: | GATCCCACTGTTAATTAAAGCTACCGTTGAAC |
| | (SEQ ID NO: 11) | 58981284-58981333 | TTACTGTTTAATGATTTC |
| | SP4 | | GAAATCATTAAACAGTAAGTTCAACGGTAGCT |
| | (SEQ ID NO: 12) | | TTAATTAACAGTGGGATC-Biotin |
| Two | Sequence 5 | chr5: | CCCAGCTCAGGCTCCACCGTGGTTACATGACG |
| 5hm | (SEQ ID NO: 13) | 111327335-111327384 | ACACAAATGAGAAATGCT |
| C | SP5 | | AGCATTTCTCATTTGTGTCGTCATGTAACCAC |
| | (SEQ ID NO: 14) | | GGTGGAGCCTGAGCTGGG-Biotin |
| | Sequence 6 | chr3: | TGGGCTAGGGCAAGCACTTCGGGGAGAGGTAC |
| | (SEQ ID NO: 15) | 53062888-53062937 | GAGAGGGAACAAAGGCAT |
| | SP6 | | ATGCCTTTGTTCCCTCTCGTACCTCTCCCCGA |
| | (SEQ ID NO: 16) | | AGTGCTTGCCCTAGCCCA-Biotin |
| Three | Sequence 7 | chr12: | CTGTGACAGCAGAAAGCGCTGCGTACCTCCCA |
| 5hm | (SEQ ID NO: 17) | 58029026-58029075 | ACGACCTTTCACCAAAGA |
| C | SP7 | | TCTTTGGTGAAAGGTCGTTGGGAGGTACGCAG |
| | (SEQ ID NO: 18) | | CGCTTTCTGCTGTCACAG-Biotin |
| | Sequence 8 | chr4: | CATCGCAGCTTTCCCACGATGGCTGCCGATTA |
| | (SEQ ID NO: 19) | 153978392-153978441 | GCCGAGGTGCGCGTTGGA |
| | SP8 | | TCCAACGCGCACCTCGGCTAATCGGCAGCCAT |
| | (SEQ ID NO: 20) | | CGTGGGAAAGCTGCGATG-Biotin |

*Underlined C represents 5hmC modifications.

### Example 5. Application of SMEL to cfDNA

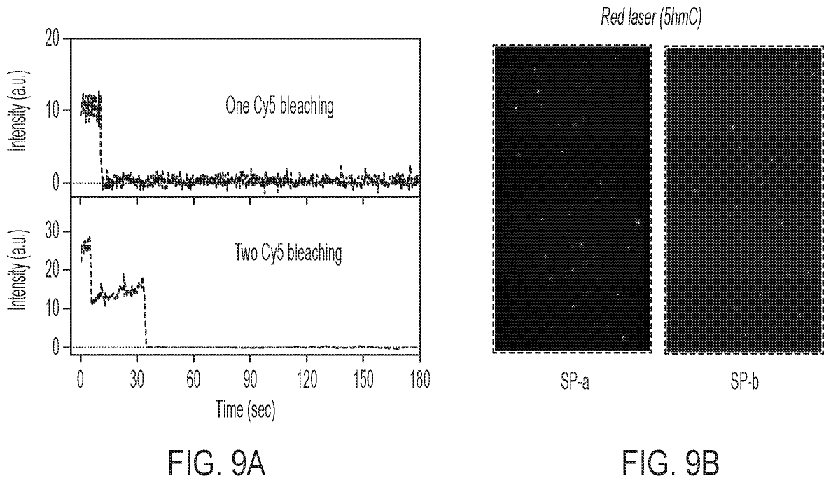

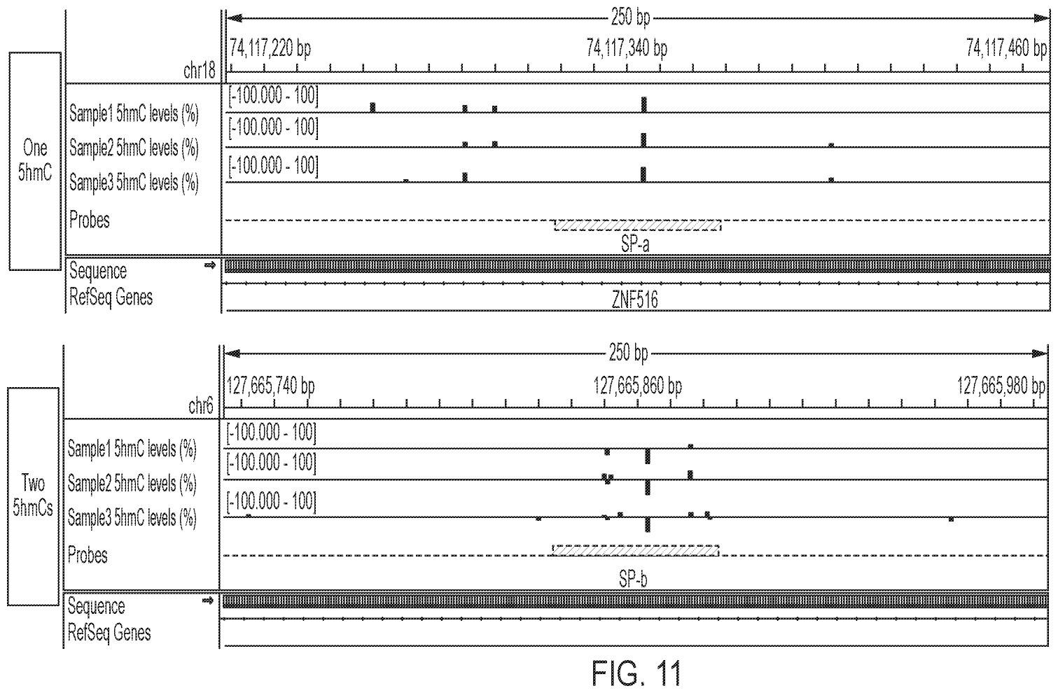

The ultra-low input requirement enables SMEL to be applicable to limited and sensitive samples, such as cfDNA from human peripheral blood. Based on cfDNA 5hmC sequencing, especially the recently reported base-resolution sequencing, probes SP-a,b were designed to target single (one) or double (two) 5hmC modifications, respectively. SMEL was then applied to cfDNA from healthy individuals (FIG. 3A, Table 3, and FIG. 11). In addition to counting fluorophores, the number of traces with single or multiple photo-beaching was calculated (FIGS. 9A-9B). For SP-b, 11.43% showed two-step Cy5 photo-bleaching compared to only 1.30% for SP-a (FIG. 3D and Table 4). Results show the disclosed single-molecule optical imaging technique is suitable for use with minute amounts of cfDNA. SMEL makes it possible to determine the specific genome location of 5hmC provides an imaging tool for using epigenetic modifications of cfDNA for cancer diagnosis.

TABLE 3
5hmC-harbored human cfDNA and corresponding SP
(single-stranded probe) sequences

| Name | Position | Sequence (5′-3′) |
|---|---|---|
| Sequence a | chr18: | CACTGCACACACCCACCAGTGCTACCCGCA |
| (One 5hmC) | 74117319-4117368 | TAGGACAGGACACTCAGGAA |
| (SEQ ID NO: 21) | | |
| SP-a | | TTCCTGAGTGTCCTGTCCTATGCGGGTAGC |
| (SEQ ID NO: 22) | | ACTGGTGGGTGTGTGCAGTG-Biotin |
| Sequence b | chr6: | TCCGTATCGTAAAACTATCCTCCCTGTTCG |
| (Two 5hmC) | 127665835-27665884 | GCGCGTTGGCACATTCTGTT |
| (SEQ ID NO: 23) | | |
| SP-b | | AACAGAATGTGCCAACGCGCCGAACAGGGA |
| (SEQ ID NO: 24) | | GGATAGTTTTACGATACGGA-Biotin |

*Underlined C represents 5hmC modifications.

TABLE 4
Number of DNA molecules
analyzed in FIGS. 3c and 3d

| | | One | Two | Three |
|---|---|---|---|---|
| Name | N | 5hmC | 5hmC | 5hmC |
| SP3 | 273 | 0.9963 | 0.0037 | 0 |
| SP4 | 240 | 0.9958 | 0.0042 | 0 |
| SP5 | 225 | 0.8578 | 0.1422 | 0 |
| SP6 | 236 | 0.8432 | 0.1568 | 0 |
| SP7 | 126 | 0.7937 | 0.1746 | 0.0317 |
| SP8 | 129 | 0.7907 | 0.1783 | 0.0310 |
| SP-a | 232 | 0.9870 | 0.0130 | 0 |
| SP-b | 210 | 0.8857 | 0.1143 | 0 |

### Example 6. Diagnosis of Cancer by SMEL Analysis

First, single-stranded DNA probes complementary to known genomic regions containing epigenetic modifications associated with a type of cancer are designed. A sample containing cfDNA is obtained from a patient suspected of having the type of cancer. The cfDNA is labeled and imaged according to the disclosed SMEL methods to localize DNA epigenetic modifications in the patient's cfDNA and generate an epigenetic profile. The patient's epigenetic profile is compared to a reference epigenetic profile for the type of cancer assessed. When the patient's epigenetic profile and the reference epigenetic profile are substantially concordant, the patient is diagnosed with cancer. The method may further be used to assess progress and stage of cancer, using external and internal controls.

Patents, applications, and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A method for localizing epigenetic modifications of DNA, the method comprising:
(a) providing a target DNA strand comprising a least one epigenetic modification, wherein the target DNA strand is annealed to a non-target DNA strand, wherein each of the target DNA strand and the non-target DNA strand is labeled with a first fluorophore at a 3′ end;
(b) labeling the at least one epigenetic modification with a second fluorophore;
(c) annealing a first probe to the target DNA strand and annealing a second probe to the non-target DNA strand;
(d) immobilizing the target DNA strand on a support; and
(e) detecting the first and second fluorophores immobilized on the support.

2. The method according to claim 1, wherein the first and second fluorophores are selected from the group consisting of Cy3, Cy5, Quasar 570, Quasar 670, Alexa Fluor 555, Alexa Fluor 647, BODIPY V-1002, BODIPY V-1005, POPO-3, TOTO-3, PO-PRO-3, and TO-PRO-3.

3. The method according to claim 2, wherein the first fluorophore is optically-distinguishable from the second fluorophore.

4. The method according to claim 1, wherein the epigenetic modification is selected from the group consisting of 5-hydroxymethylcytosine (5hmC), 5-methylcytosine (5mC), 5-formylcytosine (5fC), and 5-carboxylcytosine (5caC).

5. The method according to claim 1, wherein the first and second probes are single stranded and complementary to each other.

6. The method according to claim 1, wherein the first and second probes are labeled with a biotin moiety and the support comprises a surface-tethered moiety selected from the group consisting of avidin, streptavidin, and neutravidin.

7. The method according to claim 1, wherein the target DNA strand is immobilized via avidin-biotin pairing.

8. The method according to claim 1, wherein the support comprises a polymer-coated quartz surface.

9. The method according to claim 1, wherein the DNA is selected from the group consisting of genomic DNA and cell-free DNA (cfDNA).

10. The method according to claim 1, wherein detecting comprises imaging via prism-based single molecule total internal reflection fluorescence (TIRF) microscopy.

11. The method according to claim 10, wherein the imaging provides loci-specific localization of at least one epigenetic modification.

12. The method according to claim 10, wherein the imaging provides strand-specific localization of at least one epigenetic modification.

13. The method according to claim 1, further comprising:
incubating the product of step (c) with an exonuclease to digest non-annealed single stranded DNA prior to the immobilizing of step (d).

14. The method according to claim 13, wherein the exonuclease is E. coli Exonuclease I.

15. The method according to claim 13, wherein the method comprises an attomolar detection limit.

16. A method of diagnosing cancer in a subject suspected of having cancer, the method comprising:
(a) providing a biological sample from the subject, the sample comprising a target DNA strand comprising a least one epigenetic modification, wherein the target DNA strand is annealed to a non-target DNA strand;
(b) labeling the target DNA strand and the non-target DNA strand with a first fluorophore at a 3′ end;
(c) annealing a first probe to the target DNA strand and annealing a second probe to the non-target DNA strand;
(d) immobilizing the target DNA strand on a support;
(e) detecting the first and second fluorophores immobilized on the support, wherein detecting comprises imaging via prism-based single molecule total internal reflection fluorescence (TIRF) microscopy, wherein the imaging provides loci-specific and strand-specific localization of at least one epigenetic modification;
(f) comparing the loci-specific and strand-specific localization to a reference epigenetic profile for cancer; and
(g) diagnosing the subject as having cancer when the imaging of step (e) correlates with the reference epigenetic profile for cancer.

17. The method according to claim 16, wherein the first and second fluorophores are selected from the group consisting of Cy3, Cy5, Quasar 570, Quasar 670, Alexa Fluor 555, Alexa Fluor 647, BODIPY V-1002, BODIPY V-1005, POPO-3, TOTO-3, PO-PRO-3, and TO-PRO-3.

18. The method according to claim 17, wherein the first fluorophore is optically-distinguishable from the second fluorophore.

19. The method according to claim 16, wherein the epigenetic modification is selected from the group consisting of 5-hydroxymethylcytosine (5hmC), 5-methylcytosine (5mC), 5-formylcytosine (5fC), and 5-carboxylcytosine (5caC).

20. The method according to claim 16, wherein the first and second probes are single stranded and complementary to each other.

21. The method according to claim 16, wherein the first and second probes are labeled with a biotin moiety and the support comprises a surface-tethered moiety selected from the group consisting of avidin, streptavidin, and neutravidin.

22. The method according to claim 16, wherein the target DNA strand is immobilized via avidin-biotin pairing.

23. The method according to claim 16, wherein the support comprises a polymer-coated quartz surface.

24. The method according to claim 16, wherein the target DNA is selected from the group consisting of genomic DNA and cell-free DNA (cfDNA).

25. The method according to claim 16, wherein detecting comprises imaging via prism-based single molecule total internal reflection fluorescence (TIRF) microscopy.

26. The method according to claim 16, further comprising:
incubating the product of step (c) with an exonuclease to digest non-annealed single stranded DNA prior to the immobilizing of step (d).

27. The method according to claim 26, wherein the exonuclease is E. coli Exonuclease I.

28. The method according to claim 26, wherein the method comprises an attomolar detection limit.

29. The method according to claim 16, wherein the biological sample is selected from the group consisting of blood, serum, plasma, urine, tissue, and cultured cells.

30. The method according to claim 16, further comprising treating the diagnosed subject with a therapeutic agent specific for the cancer.


SEQUENCE LISTING (24 sequences)

SEQ ID NO: 1 - DNA, 36 bp, Homo sapiens
cccgacgcatgatctgtacttgatcgaccgtgcaac

SEQ ID NO: 2 - DNA, 36 bp, Homo sapiens
gttgcacggtcgatcaagtacagatcatgcgtcggg

SEQ ID NO: 3 - DNA, 36 bp, Artificial Sequence; identical to SEQ ID NO: 2
  single-stranded DNA probe

SEQ ID NO: 4 - DNA, 36 bp, Artificial Sequence; identical to SEQ ID NO: 1
  single-stranded DNA probe

SEQ ID NO: 5 - DNA, 50 bp, Homo sapiens
gaaaggtggagaggcgcgcagggttacccgagtgagctccggcaccctga

SEQ ID NO: 6 - DNA, 50 bp, Artificial Sequence
  single-stranded DNA probe SP1
tcagggtgccggagctcactcgggtaaccctgcgcgcctctccacctttc

SEQ ID NO: 7 - DNA, 50 bp, Homo sapiens
gaaatgctttgcatccctctcgagcctggccatataggtaatggctttgc

SEQ ID NO: 8 - DNA, 50 bp, Artificial Sequence
  single-stranded DNA probe SP2
gcaaagccattacctatctggacaggctcgagagggacgccaagcatttc

SEQ ID NO: 9 - DNA, 50 bp, Homo sapiens
ttatcttcaaggccttcattgtgccgtcattgttagcgctttcaaccttt

SEQ ID NO: 10 - DNA, 50 bp, Artificial Sequence
  single-stranded DNA probe SP3
aaaggttgaaagcgctaacaatgacggcacaatgaaggccttgaagataa

SEQ ID NO: 11 - DNA, 50 bp, Homo sapiens
gatcccactgttaattaaagctaccgttgaacttactgtttaatgatttc

SEQ ID NO: 12 - DNA, 50 bp, Artificial Sequence
  single-stranded DNA probe SP4
gaaatcattaaacagtaagttcaacggtagctttaattaacagtgggatc

SEQ ID NO: 13 - DNA, 50 bp, Homo sapiens
cccagctcaggctccaccgtggttacatgacgacacaaatgagaaatgct

SEQ ID NO: 14 - DNA, 50 bp, Artificial Sequence
  single-stranded DNA probe SP5
agcatttctcatttgtgtcgtcatgtaaccacggtggagcctgagctggg

SEQ ID NO: 15 - DNA, 50 bp, Homo sapiens
tgggctagggcaagcacttcggggagaggtacgagagggaacaaaggcat

SEQ ID NO: 16 - DNA, 50 bp, Artificial Sequence
  single-stranded DNA probe SP6
atgcctttgttccctctcgtacctctccccgaagtgcttgccctagccca

SEQ ID NO: 17 - DNA, 50 bp, Homo sapiens
ctgtgacagcagaaagcgctgcgtacctcccaacgacctttcaccaaaga

SEQ ID NO: 18 - DNA, 50 bp, Artificial Sequence
  single-stranded DNA probe SP7
tctttggtgaaaggtcgttgggaggtacgcagcgctttctgctgtcacag

SEQ ID NO: 19 - DNA, 50 bp, Homo sapiens
catcgcagctttcccacgatggctgccgattagccgaggtgcgcgttgga

SEQ ID NO: 20 - DNA, 50 bp, Artificial Sequence
  single-stranded DNA probe SP8
tccaacgcgcacctcggctaatcggcagccatcgtgggaaagctgcgatg

SEQ ID NO: 21 - DNA, 50 bp, Homo sapiens
cactgcacacacccaccagtgctacccgcataggacaggacactcaggaa

SEQ ID NO: 22 - DNA, 50 bp, Artificial Sequence
  single-stranded DNA probe SP-a
ttcctgagtgtcctgtcctatgcgggtagcactggtgggtgtgtgcagtg

SEQ ID NO: 23 - DNA, 50 bp, Homo sapiens
tccgtatcgtaaaactatcctccctgttcggcgcgttggcacattctgtt

SEQ ID NO: 24 - DNA, 50 bp, Artificial Sequence
  single-stranded DNA probe SP-b
aacagaatgtgccaacgcgccgaacagggaggatagttttacgatacgga